(19) United States
(12) Patent Application Publication
(10) Pub. No.: US 2012/0108907 A1
(43) Pub. Date: May 3, 2012

(54) Vaginal Speculum Provided With An Automatically Actuated Illumination System

(76) Inventors: Benedito Fitipaldi, Sao Paulo (BR); Cicero Livio Omegna de Souza Filho, Campinas (BR)

(21) Appl. No.: 13/382,880

(22) PCT Filed: Feb. 24, 2010
(86) PCT No.: PCT/BR2010/000055

§ 371 Date: Jan. 6, 2012

(30) Foreign Application Priority Data
Jul. 7, 2009 (BR) MU 8901270-4

(51) Int. Cl. A61B 1/32

(52) U.S. Cl. 600/223

(57) ABSTRACT

The present invention relates to the field of gynecological instruments. A disposable speculum is provided with a cylindrical filament containing an optical fiber fitted with a small aligning wing nut (3) at the distal end thereof for fitting and coupling the optical fiber to the aperture (4) in the cap (5) of the head (6), with an automatically actuated illumination system consisting of an actuating disk (7), a spring (8), a pin (9), a lens (10), a light-emitting diode/LED (11), a circuit (12), a diffuser (13), a diffuser shell (14), a cable (15) and a power source (16) with pins (17) for connecting the device to the power grid at the site of use. The head (6) can be coated with a protecting film (18) in order to prevent direct contact with the speculum (1) and the cylindrical filament (2).

### TECHNICAL FIELD

The present invention relates to a vaginal speculum pertaining to the field of gynecological instruments, which was developed in order to allow that the procedures carried out with it could be made in a more practical, adequate and safe way than the ones using similar known instruments.

### BACKGROUND

According to general knowledge, specula are instruments used in gynecological medicine for examining the inner womb region and carrying our several procedures such as cauterization and material collection for analysis, among others. They are basically comprised by two symmetrical spindles which are elongated and hinged to one another by means of screws, trammel systems or other similar devices.

In order to improve the visibility of the site and enable better operating conditions for the physician or health professional, some models are coupled to illumination devices comprised by cables having an electrical plug, switch and a remote lamp, configured to be inserted and kept in the inner portion of the speculum throughout the carrying out of the procedures. As prior art, there is also a speculum comprising a cylindrical longitudinal salience, which runs through the inner portion of one of its spindles in whose inner part an optical fiber cable is inserted that is fed by a luminous source comprising an on/off switch and which is battery powered.

It was noticed, however, that the presence of manual on/off switches can result in some drawbacks, such as forgetting and leaving them on and, therefore, resulting in an increase of the expenditure of electricity and the decrease of the life span of the lamps and of the batteries that are eventually used as the power source as well. Besides this, an eventual need of handling the switch after the adequate dressing of the professional who will carry out the procedures, could be a contamination risk for the patient that is being assisted.

Another delicate issue refers to the adequate cleaning and “isolation” of the illumination elements for the purpose of meeting the demands for safety and health of the operations, as the electrical devices must be cleaned and sterilized with extreme care. In all the models belonging to the prior art, the luminous sources get in direct contact with the speculum, which increases the contamination risks and, therefore, constitutes an inadequate condition for the procedure.

Besides that, the existence of optical fiber ends up introducing an additional step to the production procedure which, on its end, makes the final product more expensive.

### SUMMARY OF THE INVENTION

Having such drawbacks in sight and with the objective of solving them, the improvement to a vaginal speculum provided with an automatically actuated illumination system was developed, which is the object of the present patent, comprising a disposable speculum with a cylindrical filament which acts as the guide to the light (optical guide) for the reflection and propagation of the light issued by a luminous source placed in the inner portion of a head having a light-emitting diode (led), whose actuation is done automatically when the device is coupled to the cylindrical filament.

The illumination system used comprises a power source to be connected to the power grid, a cable for the transmission of the electrical power to the led, and a head comprising a compartment which houses the led and has a central aperture for the coupling of the cylindrical filament/optical guide, which is provided with a small aligning wing nut that, besides keeping the head secured to the speculum, is also responsible for putting pressure of a metal plate existing in its inner portion and that enables the necessary electrical contact for the turning on of the light. Therefore, when the head of the illumination system is dismounted from the speculum, the inner lamp is turned off automatically and immediately,

The illumination of the inner portion of the speculum and, therefore, of the action field, happens through the transport of light made by the very plastic that the surgical instrument is made of (crystal, for example), which transforms into a guide of light for being surrounded by air that has a refraction index smaller enough than the one of the material of the instrument, an optical fiber for example, which has a transparent material with a refraction index greater in its core than on its shell. Thus, the very cylindrical filament acts as the light guide (optic guide) without using the optical fiber normally used on similar products present in the art, which greatly reduces the cost of the product.

The device was designed to shed a white and intense light with a color temperature between 4500 and 5000 degrees Kelvin, which means a white light with a strong blue component that aids the vision in the observations of depth, enabling a better contrast of the red color. Besides that, the device will also benefit procedures which involve the use of colposcopes which comprise filament lamps with halogen gases, enabling a light with 3200° K approximately (a more red light). Therefore the illumination of the field with the aid of the speculum provided by the herein presented device, shall be of great use for the professional.

As far as safety and healthfulness of the operation are concerned, the configuration of the optic guide of the speculum as well as of the illumination head enable the coating of the later with a protecting film without the loss of electrical connection so the device never enters in direct contact with the speculum, thus minimizing the risks of contamination.

One notices, therefore, that the invention possesses new functional features that solve the drawbacks observed in the current prior art, besides introducing a greater safety, economy and agility to the procedures.

### BRIEF DESCRIPTION OF THE DRAWINGS

The object shall be better described and detailed based on the attached drawings:

FIG. 1 shows a perspective view of the speculum and the illumination system separately and ready for coupling;

FIG. 2 shows the coupling procedure of the parts;

FIGS. 3.1, 3.2 and 3.3 show, in amplified detail, the way of coupling and fitting in of the speculum with the head;

FIG. 4 shows the illumination head duly wrapped by the protecting film;

FIG. 5 shows the illumination system in an exploded perspective view with its comprising parts shown separately;

FIG. 6 shows a schematic partial perspective section view of the head, and

FIGS. 7.1 and 7.2 show a schematic partial transversal section view of the head, actuating disk and the top portion of the spring, and

FIG. 7.2 shows its configuration after the coupling of the cylindrical filament/optic guide which enables the contact between the actuating disk and the spring for turning on the illumination system.

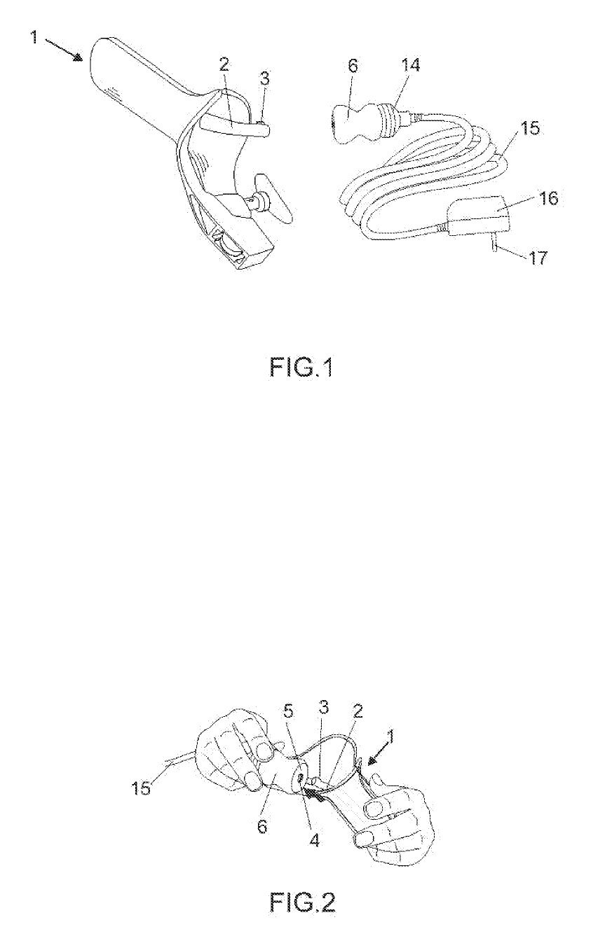

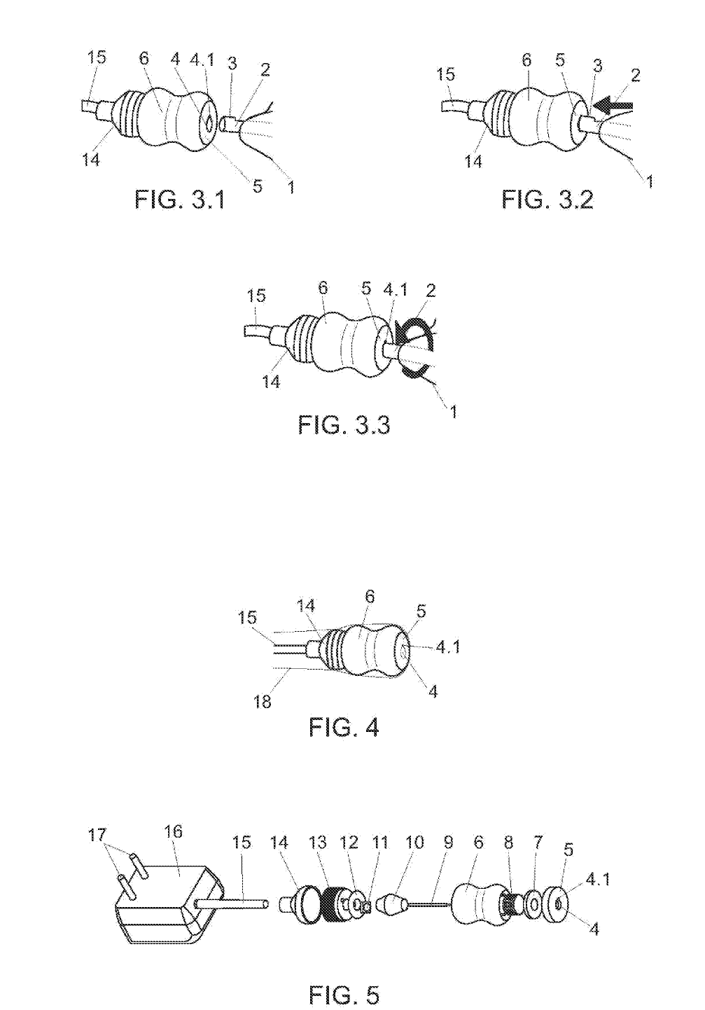

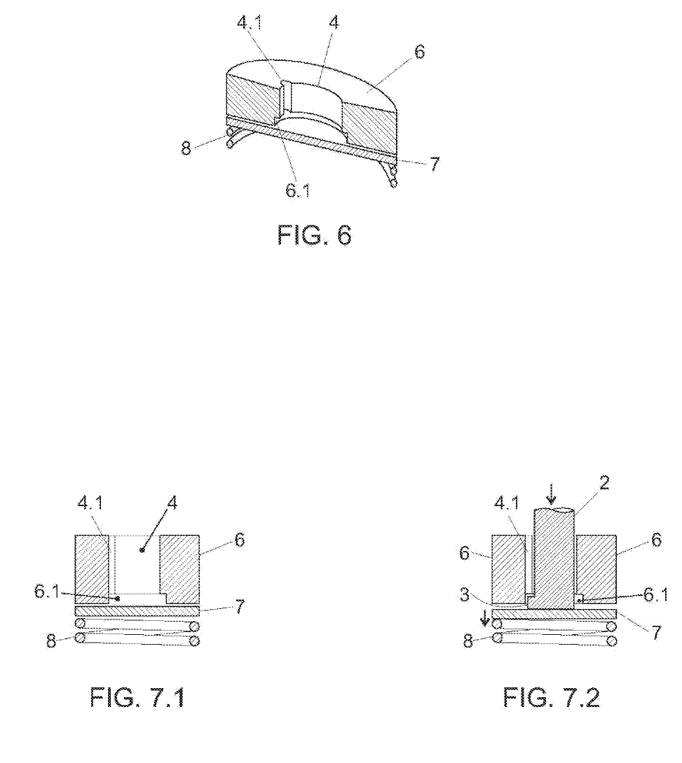

### DETAILED DESCRIPTION OF THE INVENTION

With reference to the drawings, an improved vaginal speculum provided with an automatically actuated illumination system comprises: a disposable speculum (1) provided with a cylindrical filament containing an optical fiber (2) fitted with a small aligning wing nut (3) at the distal end thereof for fitting and coupling the optical fiber to the aperture (4) in the cap (5) of the head (6) of the illumination system, consisting of an actuating disk (7), a spring (8), a pin (9), a lens (10), a light-emitting diode/LED (11), a circuit (12), a diffuser (13), a diffuser shell (14), a cable (15) and a power source (16) with pins (17) for connecting the device to the power grid at the site of use. Such a head (6) can be coated with a protecting film (18) in order to prevent direct contact with the speculum (1) and the cylindrical filament (2), besides also protecting the illumination system against secretions or lubricants eventually present on the hands of the operator, which could damage the correct functioning of the electric components. It is important to mention that the presence of the protecting film (18) does not interfere with the electric connections or contacts necessary for the correct functioning of the system.

In order to use the device, the user must initially “dress” the head (6) with the protecting film (18) and, after that, make the connection between the vaginal speculum (1) and the illuminator, which shall be carried out mechanically by fitting the cylindrical filament (2) of the speculum (1) and its small aligning wing nut (3) in the aperture (4) of the head (6). After the insertion of the filament (2) in the corresponding aperture (4), one should make a mild compression against the internal disk (7) and make a slight circular move of any of the parts in any direction for the coupling of the filament (2) to the head (6). The turning on of the system is effected by the mere compression of the filament (2) against the actuating disk (7) of the head (6).

For the disassembly and disposal of the speculum (1), you need only turn again both parts—head(6) and speculum (1), in any direction for the wing nut (3) to reach the rail in the aperture (4), thus allowing the separation of the parts.

Therefore, according to what was described and shown, one concludes that the present invention meets the requirements of the objects described above.

We claim:

1-11. (canceled)

12. A vaginal speculum assembly comprising:
a disposable speculum (1) having a cylindrical filament (2) extending therefrom, the cylindrical filament (2) of the disposable speculum (1) having a small aligning wing nut (3) provided at a distal end thereof; and,
an automatically actuated illumination system having a head including an aperture for coupling to and fixing the cylindrical filament and the disposable speculum to the illumination system, the coupling of the head to the cylindrical filament automatically turning on the illumination system.

13. The vaginal speculum assembly of claim 12, wherein the cylindrical filament (2) acts as a light guide for directing illumination coming from the illumination system, the cylindrical filament being manufactured with the same plastic that the instrument is made of, crystal polystyrene (P.S.), which turns the filament into a light guide by including air that has a refraction index smaller enough than the refraction index of the P.S..

14. The vaginal speculum assembly of claim 12, wherein the coupling of the disposable speculum (1) and the illumination system is completed by insertion of the cylindrical filament (2) in the aperture (4), thereby applying a corresponding mild pressure against an internal disk (7) located in the head, then making a small circular movement of either the speculum or the head in any direction to misalign the aligning wing nut within the aperture for fixing the cylindrical filament (2) to the head (6).

15. The vaginal speculum assembly according to claim 1, wherein the fitting of the aligning wing nut (3) within the aperture in the head guarantees the position of the cylindrical filament (2) inside the illumination head (6).

16. The vaginal speculum assembly according to claim 1, wherein the head (6) has a cap (5) with an aperture (4), an actuating disk (7) disposed in the head in communication with the aperture and being adjacent a spring (8) the cylindrical filament being engageable with the actuating disk for automatically turning on the illumination system.

17. The vaginal speculum assembly according to claim 1 wherein the illumination system includes a pin (9), a lens (10), a light-emitting diode/LED (11), a circuit (12), a diffuser (13), a diffuser shell (14), a cable (15) and a power source (16) with pins (17) for connecting the illumination system to a power grid at a site of use.

18. The vaginal speculum assembly according to claim 16, wherein the illumination system is automatically actuated by a compression of the actuating disk (7) against the spring (8).

19. The vaginal speculum assembly according to claim 1, further comprising a protecting film (18) for placement over the head (6) for protecting the illumination system during use.

20. The vaginal speculum assembly according to claim 1, wherein the illumination system has a light source which emits a white and intense light with a color temperature between 4500 and 5000 degrees Kelvin.

21. The vaginal speculum assembly according to claim 1, wherein the aligning wing nut (3) on the distal end of the cylindrical filament (2) is coupled within the aperture (4) of the head (6) when received within an internal transversal guide (6.1).

22. The vaginal speculum assembly according to claim 21, wherein the aligning wing nut (3) passes through a channel (4.1) existing in the aperture (4) which leads to the internal transversal guide (6.1), such that rotating either the cylindrical filament or the head moves the aligning wing nut from the channel into the internal transversal guide, fixing the cylindrical filament to the head.

23. The vaginal speculum assembly according to claim 16, wherein an electrical contact is made when the distal end of the cylindrical filament (2) pushes the actuating disk (7) into contact with the spring (8).
